(19) United States
(12) Patent Application Publication
(10) Pub. No.: US 2026/0108387 A1
(43) Pub. Date: Apr. 23, 2026

(54) CONFIGURABLE ULTRASONIC TRANSDUCER

(71) Applicant: Johnson & Johnson Surgical Vision, Inc., Irvine, CA (US)

(72) Inventor: Mark Delsman, Irvine, CA (US)

(21) Appl. No.: 18/920,813

(22) Filed: Oct. 18, 2024

(51) Int. Cl. A61F 9/007

(52) U.S. Cl. CPC A61F 9/00745

(57) ABSTRACT

An ultrasonic transducer is described having a resonant horn and piezoelectric elements arranged to impart vibration to the horn. The piezoelectric elements are wedge-shaped with respect to a central axis of the horn and arranged on the horn to impart to a distal end of the horn in a longitudinal direction vibration with respect to the central axis and/or a transverse direction vibration with respect to the central axis. Configurations are also described having additional wedge-shaped piezoelectric elements that may impart vibration in a second transverse direction with respect to the central axis. A phacoemulsification probe is described including wedge-shaped piezoelectric elements. Methods for energizing the piezoelectric elements are also described.

### FIELD OF INVENTION

The present invention relates to medical systems, and in particular, but not exclusively to, a phacoemulsification apparatus and associated method.

### BACKGROUND

A cataract is a clouding and hardening of the eye's natural lens, a structure which is positioned behind the cornea, iris and pupil. The lens is mostly made up of water and protein and as people age these proteins change and may begin to clump together obscuring portions of the lens. To correct this obscuration, a physician may recommend phacoemulsification cataract surgery. Before the procedure the surgeon numbs the area with anesthesia. Then a small incision is made in the cornea of the eye. Fluids such as ophthalmic viscoelastic devices (OVDs) are injected into eye through this incision to support/protect the internal structures. The anterior surface of the lens capsule is then removed to gain access to the cataract/lens. The surgeon then uses a phacoemulsification handpiece having a titanium needle. The tip of the needle vibrates at ultrasonic frequency to sculpt and emulsify the cataract while one or more pumps aspirate lens particles and fluid from the eye through the tip. The pump may be controlled with a microprocessor. The pump may be a flow based pump, e.g., a peristaltic or progressive cavity pump, or a vacuum based pump, e.g., a venturi pump. Aspirated fluids are replaced with a balanced salt solution to maintain the anterior chamber of the eye. After removing the cataract with phacoemulsification, the softer outer lens cortex is removed with suction. An intraocular lens (IOL) is introduced into the empty lens capsule via an insertion system and the IOL unfolds. Small struts called haptics may hold the IOL in place. Once correctly implanted, the IOL restores the patient's vision.

### BRIEF DESCRIPTION OF THE DRAWINGS

FIG. 1 is a side view of a phacoemulsification probe, an irrigation source and an aspiration pump;

FIG. 1A is a cross section view of a phacoemulsification probe;

FIG. 1B is a side view of a phacoemulsification transducer;

FIG. 2 is a cross section view of a phacoemulsification transducer;

FIG. 3 is a side view of a phacoemulsification transducer;

FIG. 4 is a side view of a phacoemulsification transducer;

FIG. 4A is a top view of a split electrode pair;

FIG. 5 is a diagram of piezoelectric transducer elements expanding and contracting;

FIG. 6 is an isometric drawing of a piezoelectric element according to an aspect of the invention;

FIG. 7 is an isometric drawing of a further piezoelectric element according to an aspect of the invention;

FIG. 8 is a side view of a phacoemulsification transducer according to an aspect of the invention;

FIG. 9 is a cross section view of a phacoemulsification transducer according to an aspect of the invention;

FIG. 10 is a sideview of a phacoemulsification transducer according to an aspect of the invention;

FIG. 11 is an isometric drawing of a phacoemulsification transducer according to an aspect of the invention;

FIG. 12 is a sideview of a phacoemulsification transducer according to an aspect of the invention;

FIG. 13 is a sideview of a further phacoemulsification transducer according to an aspect of the invention;

FIG. 14 is a sideview of a further phacoemulsification transducer according to an aspect of the invention; and

FIG. 15 is a sideview of a further phacoemulsification transducer according to an aspect of the invention.

### DETAILED DESCRIPTION

An apparatus and method are provided for phacoemulsification. An apparatus is further provided for piezoelectric actuation of longitudinal, transverse and combined longitudinal/transverse vibration in a Langevin horn.

In a conventional phacoemulsification handpiece, a complex arrangement of components operates harmoniously to facilitate the delicate process of cataract removal. At the core of the phacoemulsification handpiece is a hollow resonating horn, which may be termed a Langevin transducer horn, with a hollow needle coupled with it. This assembly is fluidly coupled with an aspiration channel, which suctions away emulsified material and fluid through the center of the horn.

Encasing the aspiration channel is a housing that typically includes an irrigation portion. This segment enables the introduction of fluid around the needle and into the patient's eye, ensuring optimal conditions for the surgical process.

The resonating horn comprises a series of piezoelectric elements, which may be made of ceramic materials. These elements, which may be organized in a stack formation, encircle the horn, positioned in close proximity to one another. When subjected to an oscillating voltage, these piezoelectric components expand and contract in rapid succession. This dynamic movement induces swift longitudinal vibrations within the horn, transmitting similar oscillations to the attached needle positioned at the horn's distal end.

These longitudinal vibrations are the crux of the operation, serving as the primary mechanism for emulsifying the cataractous lens. By harnessing ultrasonic frequencies, the vibrating needle effectively breaks down the cataract, facilitating its subsequent removal with precision and efficiency. Thus, through the orchestrated interplay of these intricate components, the phacoemulsification handpiece empowers surgeons to undertake delicate ocular procedures with accuracy and efficacy.

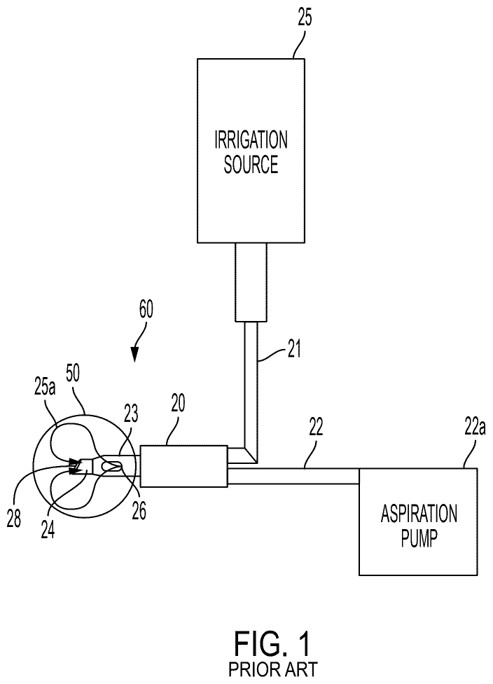

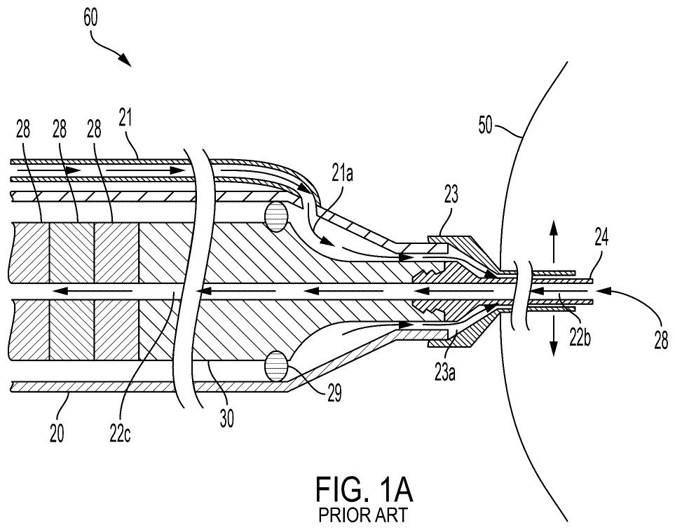

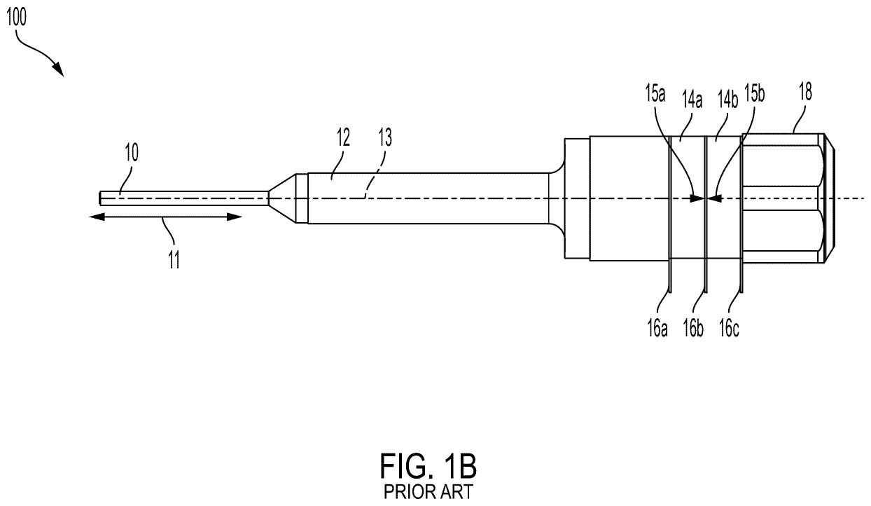

FIG. 1 is a side view of an example phacoemulsification probe 60, irrigation source 25 and aspiration pump 22a. The probe 60 includes a handpiece 20, a needle 24, a sleeve 23 having an irrigation port 26, an irrigation line 21, and an aspiration line 22. Irrigation port 26 is fluidly coupled with the irrigation line 21 to receive fluid flow from irrigation source 25. Needle 24 includes an aspiration port 28 that is fluidly coupled with the aspiration line 22 and aspiration pump 22a to receive fluid and/or material flow from eye 50. In the view shown, curved arrows in eye 50 show a typical flow of fluid 25a from irrigation source 25 into eye 50 and out through aspiration port 28.

FIG. 1A is a cross section view of a further example phacoemulsification probe 60. Corresponding elements described above with respect to FIG. 1 are labeled in FIG. 1A. Sleeve 23 is shown at the distal end of the handpiece 20, surrounding at least a portion of the needle 24 so as to expose the distal tip of the needle 24. Irrigation pathway 23a is created by the external wall of needle 24 and inner wall of sleeve 23, thereby extending the irrigation pathway 23a to the distal end of the needle 24. The needle 24 and a portion of the sleeve 23 are inserted through an incision in the cornea of the eye 50 to reach the cataractous lens. Irrigation flow 21a is supplied via irrigation line 21 through irrigation pathway 23a to port 26 (not shown) at the distal end of sleeve 23. Aspiration flow 22b enters via aspiration port 28 in the tip of the needle 24 and proceeds via aspiration channel 22c. Also shown in FIG. 1A are piezoelectric elements 28 and gasket 29, which isolates the transducer horn 30 from the handpiece 20.

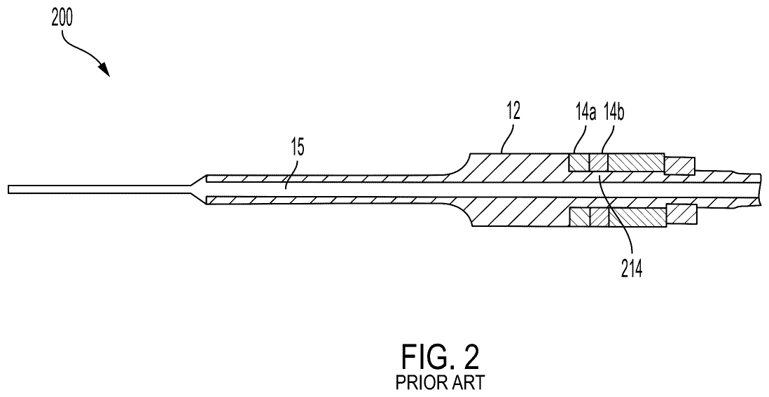

FIG. 1B is a side view of an example phacoemulsification transducer 100. The transducer 100 includes a needle 10, a horn 12, two piezoelectric elements (14a, 14b), three electrodes (16a, 16b, 16c), and a preload nut 18. FIG. 2 is a longitudinal cross-section view of a phacoemulsification transducer 100 showing horn 12, an aspiration channel 15 and two piezoelectric elements 14a and 14b. The horn 12 and the piezoelectric elements 14a, 14b may be cylindrical, with the piezoelectric elements fitted over a narrow diameter section 214 of the horn 12.

The outer two electrodes 16a, 16c may be electrically connected, while the center electrode 16b may be isolated from the two outer electrodes. Polarity of the piezoelectric elements is indicated by the arrows 15a, 15b pointing at the center electrode, i.e., in examples wherein there is a single center electrode 16b the two piezoelectric elements 14a, 14b are arranged with like polarity sides face to face. The piezoelectric elements are arranged such that when a positive voltage is applied to the outer electrodes 16a, 16c, with respect to the center electrode 16b, the piezoelectric elements 14a, 14b expand (in thickness). Conversely, when a negative voltage is applied to the outer electrodes 16a, 16c with respect to the center electrode 16b, the piezoelectric elements contract. By driving the piezoelectric elements 14a, 14b with an alternating voltage waveform, the entire structure, including the horn 12, needle 10, and preload nut 18, will vibrate in a longitudinal mode, i.e. along a center axis 13 of the probe 100, as shown by the arrows 11 near the needle 10.

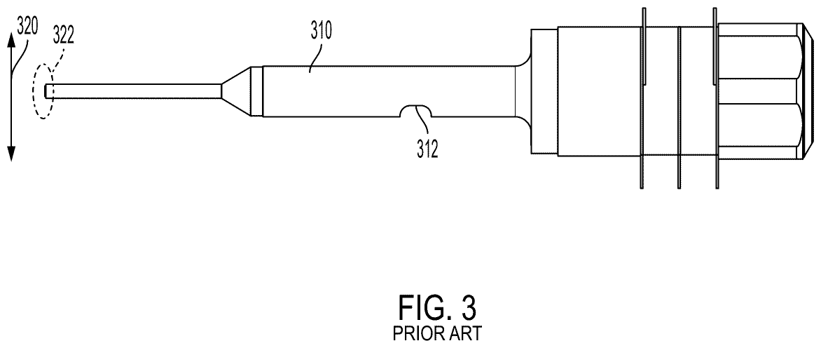

In a phacoemulsification procedure, it may be desirable to impart a motion other than a longitudinal motion to the needle 10. For example, it may be desirable to impart a transversal motion in addition to or separate from the longitudinal motion described above. With reference to FIG. 3, one way to achieve a transversal motion (i.e., side to side motion) (indicated by arrows 320) of the needle is by incorporating a mechanical imbalance in the transducer's horn structure. This is shown as a notch 312 in the long portion of the horn 310 in FIG. 3. This mechanical imbalance introduces a transversal motion component into the tip motion. Combined with the fundamental longitudinal motion, this results in an elliptical tip motion (shown as 320). A limitation of the mechanical imbalance means to introducing transversal motion is that the ratio of the longitudinal to transversal motion is fixed as it is determined by the geometry and location of the notch. As a result, the ratio of longitudinal to transversal motion of the needle cannot be varied during use.

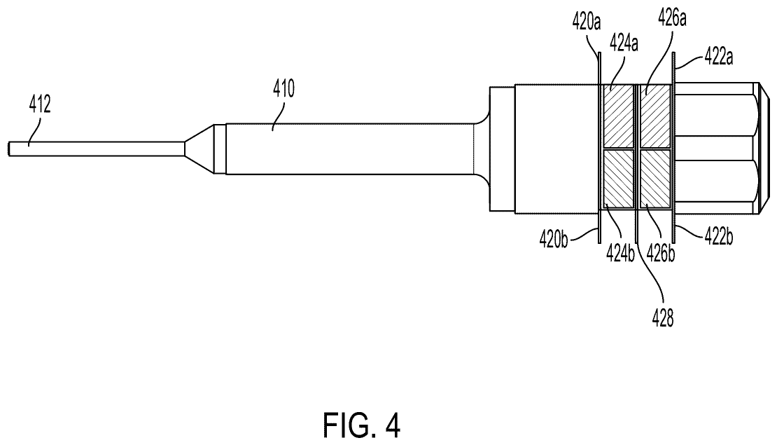

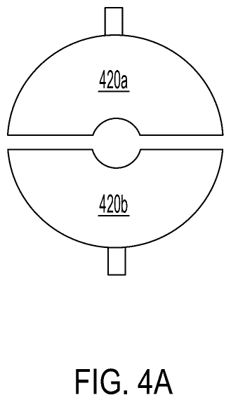

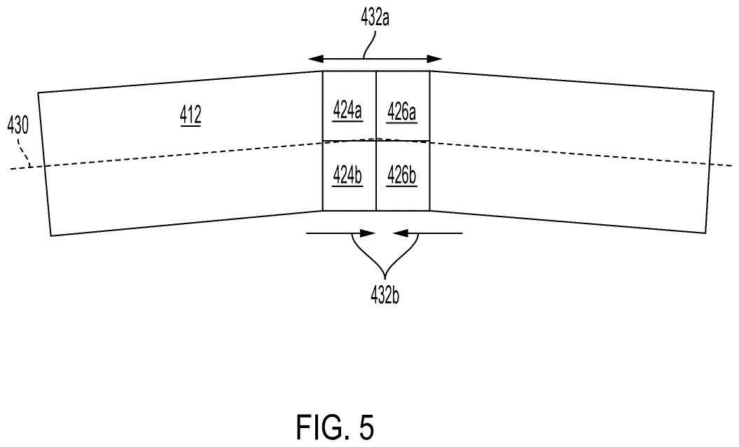

FIG. 4, shows a further means for imparting vibration via a horn 410 to a phacoemulsification needle 412. In this arrangement, the outer electrodes may be split into hemispherical halves 420a-420b and 422a-422b. A top view (i.e. as viewed in a longitudinal direction down the transducer) of an example split electrode pair 420a-420b is shown in FIG. 4A. A center electrode 428 serves as a ground in this example and is not required to be split. This enables driving the two halves of each of the piezoelectric elements independently. For example, electrodes 420a and 422a may be driven with a positive voltage with respect to ground electrode 428 (expanding piezoelectric element halves 424a and 426a), while electrodes 420b and 422b may be driven with a negative voltage (contracting piezoelectric element halves 424b and 426b). As shown in FIG. 5 (in an exaggerated illustration), this expansion on one side (as shown by arrow 432a) and contraction (as shown by arrows 432b)on the opposite side of the center axis 430 enables directly driving a bending mode in the horn 410 leading to transversal motion at the needle 412. By varying the amplitude and phasing of the drive voltage to the two piezoelectric element halves 424a/424b, 426a/426b, the needle tip motion can be varied from nearly-pure longitudinal motion (in-phase as shown e.g. in FIG. 1) to nearly-pure transversal motion (180° phase), and anything in between (e.g. elliptical).

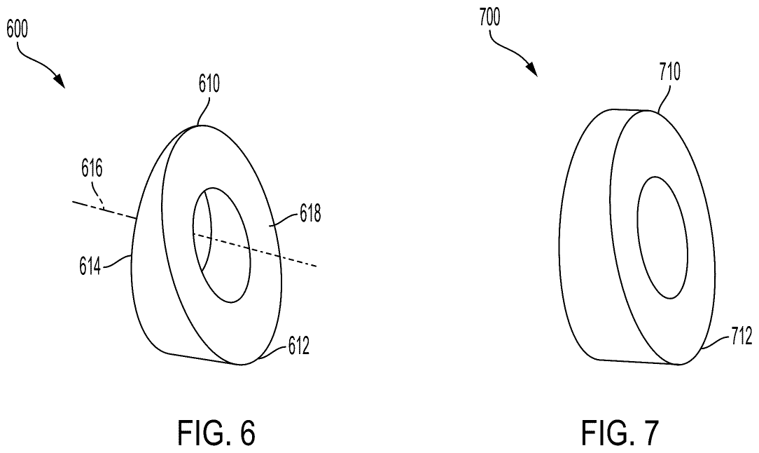

In examples as shown, e.g., FIGS. 6 and 7, a piezoelectric element 600, 700, respectively may be formed as a wedge-shaped portion (i.e., a “slice”) of a thick-walled cylinder. In examples, such a wedge-shaped piezoelectric element 600 may taper to a sharp edge 610 from a wide end 612, or a further wedge-shaped piezoelectric element 700 may taper to a thinner (but not sharp) end 710 from a wider end 712. In examples, a first face 614 of the piezoelectric element is not parallel to the second face 618 of the piezoelectric element. In further examples, the first face. 614 may be perpendicular to the center axis 616 of the piezoelectric element, while the second face 618 may lie on a plane that is not perpendicular to the center axis 616.

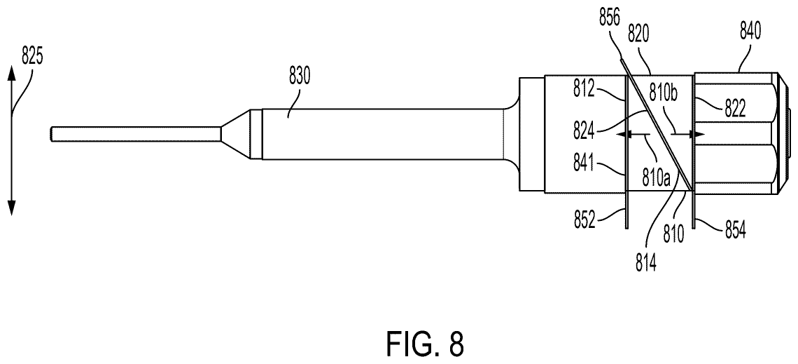

In examples as shown, e.g., FIG. 8, two wedge-shaped piezoelectric elements 810, 820 may be arranged onto a horn 830 as previously described in FIGS. 1 and 2 and accompanying descriptions with respect to simple cylinder-shaped piezoelectric elements. Solid black arrows 810a, 810b indicate polarity of the two piezoelectric elements 810, 820. A first face 812 of a first piezoelectric element 810 may be arranged in contact with an electrode 852, which in turn is backed by the horn 830. A first face 822 of a second piezoelectric element 820, may be arranged in contact with an electrode 854 and backed by a preload nut 840. The preload nut 840 urges the stack of the above-described electrodes and piezoelectric elements against a stop surface 841 of the horn 830. A second (slanted) face 814 of the first piezoelectric element 810 and a second (slanted) face 824 of the second piezoelectric element 820 may be arranged facing each other and may be each in contact with a single electrode 856. The two piezoelectric elements 810 and 820 should be arranged axially so that the thickest portion of the first piezoelectric element 810 is aligned with the thinnest portion of the second piezoelectric element. If the two slanted faces 814, 824 are not correctly aligned, then the two slanted faces will not fully meet the center electrode 856, i.e., the two slanted faces will not be parallel to each other and to the center electrode. Means for properly aligning the two piezoelectric elements 810, 820 axially, include but are not limited to: including a keyway on each piezoelectric element and/or a marking on each piezoelectric element for visual alignment. In examples, the angled electrode 856 may provide a common potential, similar to the central electrode in FIG. 1, while the outer electrodes 852, 854 may be driven independently. In a case where the outer electrodes are driven in-phase, the transducer will produce a longitudinal vibration, as both piezoelectric elements 810, 820 will expand and contract together. In a case where the outer electrodes 852, 854 may be driven 180° out of phase with each other, with respect to the center electrode 856, then one piezoelectric element (e.g. 810) will expand while the other (e.g. 820) contracts, thereby producing a transverse motion at the needle tip as shown by the arrows 825.

The outer electrodes may also be driven at phase angles other than 180° with respect to each other, as described in more detail below. Thus, the two wedge-shaped piezoelectric elements may provide a more simplified means for producing and controlling both longitudinal and transversal needle movement with less components and fewer electrical connections than the split electrode arrangement shown in FIG. 4 and then the mechanically induced imbalance of a notch in FIG. 3.

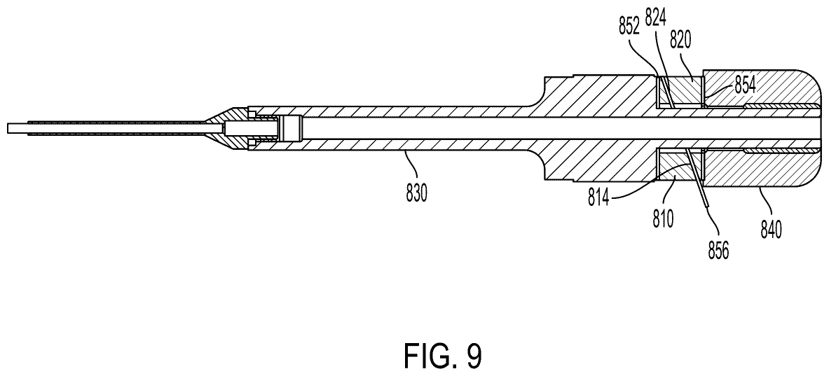

FIG. 9 is a cross section view showing the two wedge shaped piezoelectric elements as described above with respect to FIG. 8.

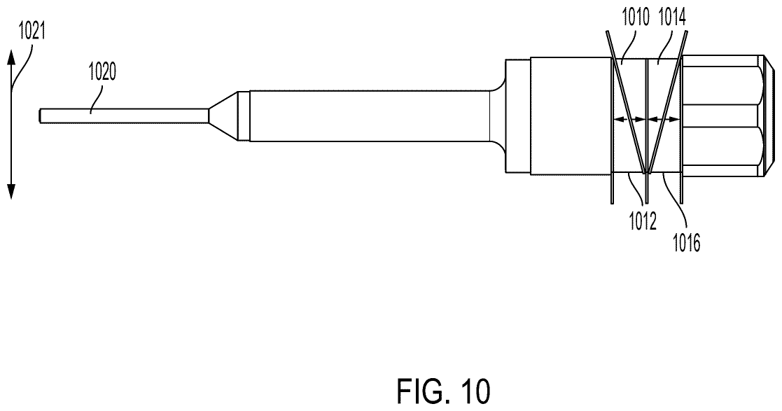

In examples, there may be arranged on a horn more than two wedge-shaped piezoelectric elements. FIG. 10 shows an exemplary probe with four wedge-shaped piezoelectric elements, 1010, 1012, 1014, 1016. In this example, element pairs 1010, 1012 and 1014, 1016 are arranged. In examples, this arrangement may enable a larger amount of transverse motion (as shown by the arrows 1021) at the needle tip 1020 than would be possible with a single pair of similar thickness wedge-shaped piezoelectric elements. In further examples, a larger number of thinner ceramics of an overall total thickness similar to a single thicker pair may reduce stresses inside the ceramic elements, and/or may improve impedance matching to an amplifier. By exciting both piezoelectric element pairs 1010/1012 and 1014/1016 with 180° out of phase excitation signals, both pairs of elements will expand and contract together at the same sides of the device, e.g. both top sides expanding while both bottom sides are contracting, thus creating the same effect as described for FIG. 8 with two piezoelectric elements but with the potential benefits over a single pair of elements in the reduction of internal stress and impedance matching described above.

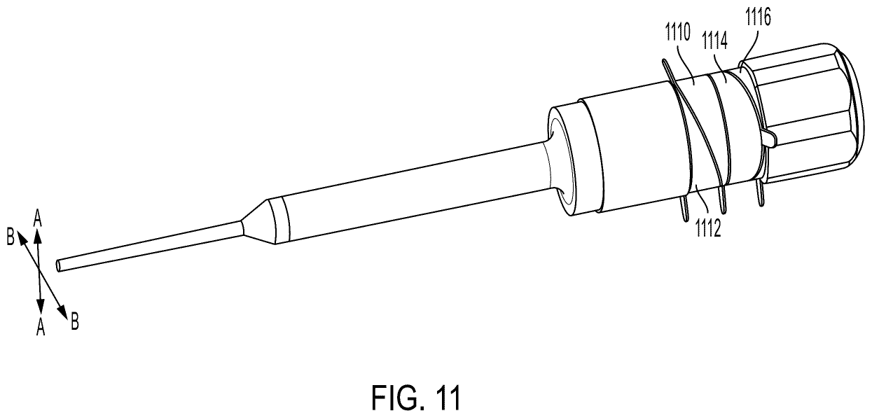

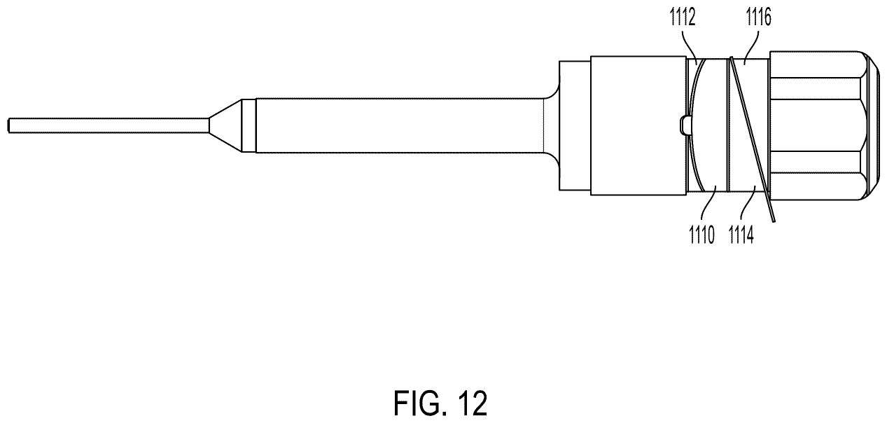

In further examples as shown, for example, in FIGS. 11 and 12, two pairs of wedge-shaped piezoelectric elements 1110, 1112/1114, 1116 may be arranged to create vibration at the needle tip in orthogonal directions. When each pair of piezoelectric elements is excited with opposite polarity signals, piezoelectric element pair 1110, 1112 creates vibration in the direction labeled A-A and piezoelectric element pair 1114, 1116 creates vibration in the direction labeled B-B, thus creating orthogonal transverse motion at the needle end. Alternating current signal combinations for exciting the two piezoelectric element pairs 1110, 1112/1114, 1116 may be arranged to create not only tip motion in first or second orthogonal directions as shown, but any combination of the two directions as well, in addition to longitudinal, allowing for circular, elliptical, and helical patterns of needle tip vibration. Other combinations of wedge-shaped elements are contemplated, and the examples disclosed herein are examples and not to be considered limiting the scope of the invention. For example, three pairs of wedge-shaped piezoelectric elements may be arranged to create transverse motion at 120° angles to each other. Wedge-shaped elements may be combined with parallel-faced elements in examples, wherein the parallel-faced elements generate longitudinal needle tip motion while the wedge-shaped elements generate transverse needle tip motion.

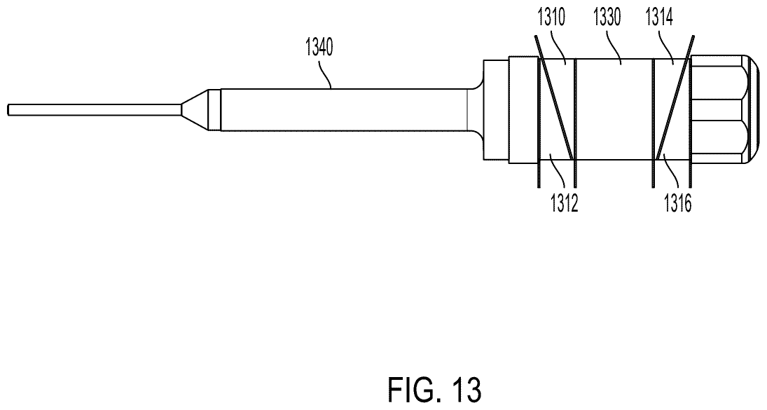

In examples, the location of the piezoelectric elements may be positioned to optimize the bending and longitudinal resonance of the transducer, as shown in FIG. 13, which example includes the addition of a central mechanical element 1330 between two wedge-shaped piezoelectric element pairs: 1310/1312, 1314/1316. In examples, the piezoelectric element pairs may be centered near both bending and longitudinal nodes of the horn 1340 at the desired drive frequency.

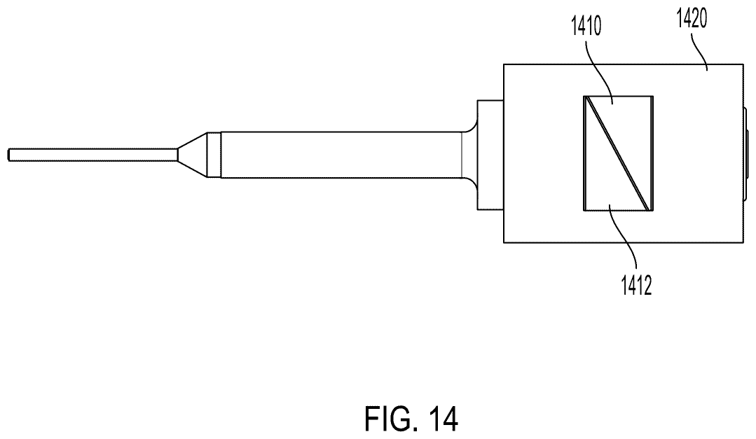

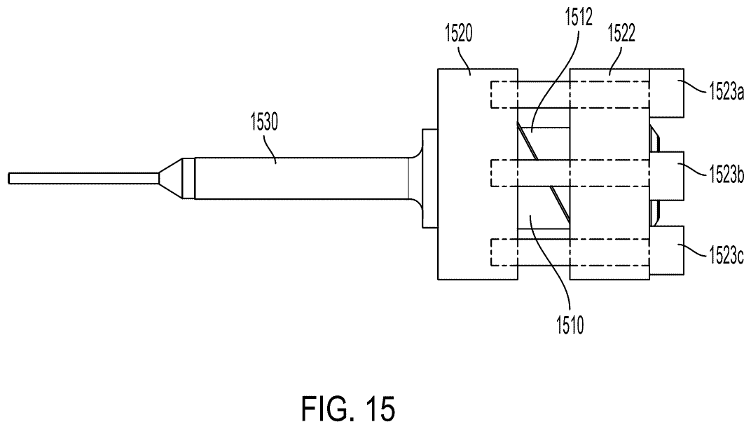

In further examples, the piezoelectric elements are arranged with an external means of preloading, such as is shown in FIG. 14, as opposed to the central core and preload nut described above. Rather than using a central preload lug, in examples, the transducer may be preloaded around the exterior, by placing the piezoelectric elements 1410, 1412 within a rigid structure 1420, as shown. FIG. 15 shows a further example of preloading piezoelectric elements 1510, 1512 by using a plurality of external preload fasteners 1523a, 1523b, 1523c. The external preload fasteners may pass through and affix a first compression element 1522 to a second compression element 1520. The second compression element may be an integral part of the transducer horn 1530 or may be a separate element that is affixed to the horn 1530. The second compression element may have internal threads to accommodate the external preload fasteners 1523a, 1523b, 1523c. Such examples, as described with respect to FIGS. 14 and 15, eliminate need for a central hole through the piezoelectric element, which may have manufacturing advantages as well as provide mechanical stability to the piezoelectric elements. In further examples, non-cylindrical components may be used. In further examples, piezoelectric elements may have square faces or other geometrical shapes.

While distinct electrode components are described herein as a means for energizing piezoelectric elements, for example as shown in FIG. 4A, it should be understood that other means for electrically coupling portions of the piezoelectric elements to driving signals are also contemplated and the invention is not limited to examples having distinct component electrodes. For example, the piezoelectric elements may have bonded contact surfaces configured for electrical connection to wire leads or other electrical contacts. In further examples, the transducer horn itself and the preload nut may each provide electrical contact directly to the piezoelectric elements.

In examples, methods of energizing wedge-shaped piezoelectric elements arranged about a resonant horn as described above are disclosed herein. In a first method, two wedge-shaped piezoelectric elements are arranged on a resonant horn having a central axis. First and second piezoelectric elements each have a first face arranged perpendicular to the central axis and a second face arranged at an acute angle to the first face, with the two piezoelectric elements arranged on the resonant horn with their angled faces facing each other and parallel to each other. First and third electrodes are arranged in contact with the respective first faces of the first and second piezoelectric elements. A second electrode is arranged between the respective second faces of the first and second piezoelectric elements. In examples, a first AC signal having a first frequency is applied to the first electrode with respect to the second electrode. The first AC signal is also applied in phase to the third electrode with respect to the second electrode, thereby causing the first and second piezoelectric elements to expand and contract together longitudinally along the central axis at the first frequency creating a longitudinal vibration in the horn along the central axis. In further examples, a first AC signal having a first frequency is applied to the first electrode with respect to the second electrode. A second AC signal, 180° out of phase with the first AC signal, is applied to the third electrode with respect to the second electrode, thereby causing the first piezoelectric element to expand while the second piezoelectric element contracts and vice-versa so as to create a transverse vibration at a distal end of the resonant horn, the transverse vibration being perpendicular to the central axis. It should be noted that some examples above discuss driving piezoelectric element pairs 180° out of phase with each other but any phase difference is possible. Adjusting phase differences allows for an adjustment of the ratio of longitudinal to transverse motion of the needle.

In a further example, third and fourth wedge-shaped piezoelectric elements are arranged on the horn described above, together with the first and second wedge-shaped piezoelectric elements. The third and fourth piezoelectric elements may each have a first face arranged perpendicular to the central axis and a second face arranged at an acute angle to the first face, with the two piezoelectric elements arranged on the resonant horn with their angled faces facing each other and parallel to each other. Fourth and sixth electrodes are arranged in contact with the respective first faces of the third and fourth piezoelectric elements. A fifth electrode is arranged between the respective second faces of the third and fourth piezoelectric elements. The fourth electrode may be in contact with the above-described third electrode or may be electrically isolated from the third electrode.

In further examples, a first AC signal having a first frequency is applied to the first electrode with respect to the second electrode. A second AC signal out of phase with the first AC signal is applied to the third electrode with respect to the second electrode, thereby causing the first piezoelectric element to expand while the second piezoelectric element contracts and vice-versa so as to create a transverse vibration at a distal end of the resonant horn, the transverse vibration being perpendicular to the central axis. The first AC signal is also applied to the sixth electrode with respect to the fifth electrode. The second AC signal out of phase with the first AC signal is applied to the fourth electrode with respect to the fifth electrode, thereby causing the third piezoelectric element to contract while the fourth piezoelectric element expands and vice-versa. In examples, the third and fourth piezoelectric elements are arranged with respect to the first and second piezoelectric elements so as to create additional transverse vibration at a distal end of the resonant horn, the transverse vibration being perpendicular to the central axis and in phase with the transverse motion created by the first and second electrodes.

In examples, the first, second, third and fourth piezoelectric elements may be energized by the above-stated first AC signal, all in phase.

In further examples, the third and fourth piezoelectric elements are arranged on the horn with respect to the first and second piezoelectric elements so as to create a transverse vibration at the distal end of the horn in a different direction than is created by the first and second piezoelectric elements. In examples, the first and second piezoelectric elements may be energized as described above with out of phase signals to create a first transverse motion at the distal end of the horn. The third and fourth piezoelectric elements may be energized as described above with out of phase signals to create a second transverse motion at the distal end of the horn. The first and second piezoelectric elements may be energized as described without the third and fourth piezoelectric elements being energized. The third and fourth piezoelectric elements may be energized as described without the first and second piezoelectric elements being energized. The first and second piezoelectric elements may be energized with a first pair of out of phase signals and the third and fourth piezoelectric elements may simultaneously be energized with as second pair of out of phase signals, which may be at a same amplitude as the first pair of out of phase signals or at a different amplitude. In further examples, the first pair of out of phase signals and/or the second pair of out of phase signals may be 180° out of phase.

In examples, an ultrasonic transducer may comprise: a horn having a central axis, a first diameter section and a second diameter section, a first piezoelectric element having a circular perimeter, a first face, a second face, and a first central through hole, wherein the second face of the first piezoelectric element is arranged at an acute angle to the first face of the first piezoelectric element, a second piezoelectric element having a circular perimeter, a first face, a second face, and a second central through hole, wherein the second face of the second piezoelectric element is arranged at an acute angle to the first face of the second piezoelectric element, wherein the first piezoelectric element and second piezoelectric element are arranged on the horn such that the second diameter section of the horn passes through the first central through hole and the second central through hole, and a fastener is arranged on the horn to urge the second face of the second piezoelectric element in proximity to the second face of the first piezoelectric element and to urge the first face of the first piezoelectric element in proximity to a surface of the first diameter section of the horn.

Additionally, the above-described ultrasonic transducer may comprise: a first electrical contact arranged to energize the first face of the first piezoelectric element, a second electrical contact arranged to energize the second face of the first piezoelectric element and the second face of the second piezoelectric element, and a third electrical contact arranged to energize the first face of the second piezoelectric element. Additionally, first and second piezoelectric elements may be arranged such that a first positive voltage applied to the first electrical contact with respect to the second electrical contact causes the first piezoelectric element to expand along the central axis and the first positive voltage applied to the third electrical contact with respect to the second electrical contact causes the second piezoelectric element to expand along the central axis.

Additionally, the above-described ultrasonic transducer may comprise: the first and second piezoelectric elements being arranged such that an alternating current applied in phase to both the first and third electrical contacts with respect to the second electrical contact causes the first and second piezoelectric elements to impart a longitudinal vibration to the horn along the central axis.

Additionally, the above-described ultrasonic transducer may comprise the first and second piezoelectric elements being arranged such that a first alternating current applied to the first electrical contact with respect to the second electrical contact and a second alternating current that is out of phase with the first alternating current applied to the third electrical contact with respect to the second electrical contact causes the first and second piezoelectric elements to impart a transverse vibration to the horn with respect to the central axis.

Additionally, the above-described ultrasonic transducer may comprise a third piezoelectric element having a circular perimeter, a first face, a second face, and a third central through hole, wherein the second face of the third piezoelectric element is arranged at an acute angle to the first face of the third piezoelectric element, a fourth piezoelectric element having a circular perimeter, a first face, a second face, and a fourth central through hole, wherein the second face of the fourth piezoelectric element is arranged at an acute angle to the first face of the fourth piezoelectric element, the third piezoelectric element and the fourth piezoelectric element are arranged on the horn such that the second diameter section of the horn passes through the third through hole and the fourth through hole, and the fastener further arranged to urge the first face of the third piezoelectric element in proximity to the first face of the second piezoelectric element, and to urge the second face of the fourth piezoelectric element in proximity to the second face of the third piezoelectric element.

Additionally, the above-described four piezoelectric element ultrasonic transducer may comprise: a first electrical contact arranged to energize the first face of the first piezoelectric element, a second electrical contact arranged to energize the second face of the first piezoelectric element and the second face of the second piezoelectric element, a third electrical contact arranged to energize the first face of the second piezoelectric element and the first face of the third piezoelectric element, a fourth electrical contact arranged to energize the second face of the third piezoelectric element and the second face of the fourth piezoelectric element, and a fifth electrical contact arranged to energize the first face of the fourth piezoelectric element.

Additionally, the above-described four piezoelectric element ultrasonic transducer may comprise: the first and second piezoelectric elements being arranged such that a first positive voltage applied to the first electrical contact with respect to the second electrical contact causes the first piezoelectric element to expand along the central axis; the first positive voltage applied to the third electrical contact with respect to the second electrical contact causes the second piezoelectric element to expand along the central axis; the third and fourth piezoelectric elements being arranged such that the first positive voltage applied to the third electrical contact with respect to the fourth electrical contact causes the third piezoelectric element to expand along the central axis; and the first positive voltage applied to the fifth electrical contact with respect to the fourth electrical contact causes the fourth piezoelectric element to expand along the central axis.

Additionally, the above-described four piezoelectric element ultrasonic transducer may comprise: the first and second piezoelectric elements being arranged such that a first alternating current applied to the first electrical contact with respect to the second electrical contact and a second alternating current that is out of phase with the first alternating current applied to the third electrical contact with respect to the second electrical contact causes the first and second piezoelectric elements to impart a first transverse vibration in a first direction to the horn with respect to the central axis; and the third and fourth piezoelectric elements being arranged such that the first alternating current applied to the fifth electrical contact with respect to the fourth electrode and the second alternating current that is out of phase with the first alternating current applied to the third electrical contact with respect to the fourth electrical contact causes the third and fourth piezoelectric elements to impart a second transverse vibration in the first direction to the horn with respect to the central axis.

Additionally, the above-described four piezoelectric element ultrasonic transducer may comprise: the first and second piezoelectric elements being arranged such that a first alternating current applied to the first electrical contact with respect to the second electrical contact and a second alternating current that is out of phase with the first alternating current applied to the third electrical contact with respect to the second electrical contact causes the first and second piezoelectric elements to impart a first transverse vibration in a first direction to the horn with respect to the central axis; and the third and fourth piezoelectric elements being arranged such that the first alternating current applied to the fifth electrical contact with respect to the fourth electrical contact and the second alternating current that is out of phase with the first alternating current applied to the third electrical contact with respect to the fourth electrical contact causes the third and fourth piezoelectric elements to impart a second transverse vibration in a second direction to the horn with respect to the central axis, the second direction being different than the first direction.

Additionally, an ultrasonic surgical instrument may comprise any of the above-described ultrasonic transducers and a needle coupled with a distal end of the horn.

What is claimed is:

1. An ultrasonic transducer comprising:
a horn having a central axis, a first diameter section and a second diameter section;
a first piezoelectric element having a circular perimeter, a first face, a second face, and a first central through hole, wherein the second face of the first piezoelectric element is arranged at an acute angle to the first face of the first piezoelectric element;
a second piezoelectric element having a circular perimeter, a first face, a second face, and a second central through hole, wherein the second face of the second piezoelectric element is arranged at an acute angle to the first face of the second piezoelectric element;
wherein the first piezoelectric element and second piezoelectric element are arranged on the horn such that the second diameter section of the horn passes through the first central through hole and the second central through hole; and
a fastener arranged on the horn to urge the second face of the second piezoelectric element in proximity to the second face of the first piezoelectric element and to urge the first face of the first piezoelectric element in proximity to a surface of the first diameter section of the horn.

2. The ultrasonic transducer of claim 1, further comprising:
a first electrical contact arranged to energize the first face of the first piezoelectric element,
a second electrical contact arranged to energize the second face of the first piezoelectric element and the second face of the second piezoelectric element, and
a third electrical contact arranged to energize the first face of the second piezoelectric element.

3. The ultrasonic transducer of claim 1, wherein the first and second piezoelectric elements are arranged such that a first positive voltage applied to the first electrical contact with respect to the second electrical contact causes the first piezoelectric element to expand along the central axis and the first positive voltage applied to the third electrical contact with respect to the second electrical contact causes the second piezoelectric element to expand along the central axis.

4. The ultrasonic transducer of claim 3, wherein the first and second piezoelectric elements are arranged such that an alternating current applied in phase to both the first and third electrical contacts with respect to the second electrical contact causes the first and second piezoelectric elements to impart a longitudinal vibration to the horn along the central axis.

5. The ultrasonic transducer of claim 3, wherein the first and second piezoelectric elements are arranged such that a first alternating current applied to the first electrical contact with respect to the second electrical contact and a second alternating current that is out of phase with the first alternating current applied to the third electrical contact with respect to the second electrical contact causes the first and second piezoelectric elements to impart a transverse vibration to the horn with respect to the central axis.

6. The ultrasonic transducer of claim 1, further comprising:
a third piezoelectric element having a circular perimeter, a first face, a second face, and a third central through hole, wherein the second face of the third piezoelectric element is arranged at an acute angle to the first face of the third piezoelectric element,
a fourth piezoelectric element having a circular perimeter, a first face, a second face, and a fourth central through hole, wherein the second face of the fourth piezoelectric element is arranged at an acute angle to the first face of the fourth piezoelectric element,
the third piezoelectric element and the fourth piezoelectric element are arranged on the horn such that the second diameter section of the horn passes through the third through hole and the fourth through hole, and
the fastener further arranged to urge the first face of the third piezoelectric element in proximity to the first face of the second piezoelectric element, and to urge the second face of the fourth piezoelectric element in proximity to the second face of the third piezoelectric element.

7. The ultrasonic transducer of claim 6, further comprising:
a first electrical contact arranged to energize the first face of the first piezoelectric element,
a second electrical contact arranged to energize the second face of the first piezoelectric element and the second face of the second piezoelectric element,
a third electrical contact arranged to energize the first face of the second piezoelectric element and the first face of the third piezoelectric element,
a fourth electrical contact arranged to energize the second face of the third piezoelectric element and the second face of the fourth piezoelectric element, and
a fifth electrical contact arranged to energize the first face of the fourth piezoelectric element.

8. The ultrasonic transducer of claim 7, wherein the first and second piezoelectric elements are arranged such that a first positive voltage applied to the first electrical contact with respect to the second electrical contact causes the first piezoelectric element to expand along the central axis;
the first positive voltage applied to the third electrical contact with respect to the second electrical contact causes the second piezoelectric element to expand along the central axis;
the third and fourth piezoelectric elements are arranged such that the first positive voltage applied to the third electrical contact with respect to the fourth electrical contact causes the third piezoelectric element to expand along the central axis; and
the first positive voltage applied to the fifth electrical contact with respect to the fourth electrical contact causes the fourth piezoelectric element to expand along the central axis.

9. The ultrasonic transducer of claim 7,
wherein the first and second piezoelectric elements are arranged such that a first alternating current applied to the first electrical contact with respect to the second electrical contact and a second alternating current that is out of phase with the first alternating current applied to the third electrical contact with respect to the second electrical contact causes the first and second piezoelectric elements to impart a first transverse vibration in a first direction to the horn with respect to the central axis; and
wherein the third and fourth piezoelectric elements are arranged such that the first alternating current applied to the fifth electrical contact with respect to the fourth electrode and the second alternating current that is out of phase with the first alternating current applied to the third electrical contact with respect to the fourth electrical contact causes the third and fourth piezoelectric elements to impart a second transverse vibration in the first direction to the horn with respect to the central axis.

10. The ultrasonic transducer of claim 7, wherein the first and second piezoelectric elements are arranged such that a first alternating current applied to the first electrical contact with respect to the second electrical contact and a second alternating current that is out of phase with the first alternating current applied to the third electrical contact with respect to the second electrical contact causes the first and second piezoelectric elements to impart a first transverse vibration in a first direction to the horn with respect to the central axis; and
wherein the third and fourth piezoelectric elements are arranged such that the first alternating current applied to the fifth electrical contact with respect to the fourth electrical contact and the second alternating current that is out of phase with the first alternating current applied to the third electrical contact with respect to the fourth electrical contact causes the third and fourth piezoelectric elements to impart a second transverse vibration in a second direction to the horn with respect to the central axis, the second direction being different than the first direction.

11. An ultrasonic surgical instrument comprising:
the ultrasonic transducer according to claim 1, and
a needle coupled with a distal end of the horn.

12. An ultrasonic surgical instrument comprising:
the ultrasonic transducer according to claim 6, and
a needle coupled with a distal end of the horn.

13. A method of energizing an ultrasonic transducer comprising a resonant horn and one or more pairs of wedge-shaped piezoelectric elements arranged on the horn, the method comprising:
applying a first alternating current signal to a first wedge-shaped piezoelectric element of a first pair of wedge-shaped piezoelectric elements;
applying the first alternating current signal to a second wedge-shaped piezoelectric element of the first pair of wedge-shaped piezoelectric elements.

14. The method of claim 13, wherein the first wedge-shaped piezoelectric element and the second wedge-shaped piezoelectric element are caused to expand and contract in phase with each other.

15. The method of claim 14, wherein the first wedge-shaped piezoelectric element and the second wedge-shaped piezoelectric element are caused to expand and contract out of phase with each other.

16. The method of claim 13, further comprising:
applying the alternating current signal to a first wedge-shaped piezoelectric element of a second pair of wedge-shaped piezoelectric elements;
applying the first alternating current signal to a second wedge-shaped piezoelectric element of the second pair of wedge-shaped piezoelectric elements.

17. The method of claim 16 wherein the first wedge-shaped piezoelectric element and the second wedge-shaped piezoelectric element of the first pair of wedge-shaped piezo-electric elements; and the first wedge-shaped piezoelectric element and the second wedge-shaped piezoelectric element of the second pair of wedge-shaped piezoelectric elements are all caused to expand and contract in phase with each other.

18. The method of claim 16 wherein the first wedge-shaped piezoelectric element and the second wedge-shaped piezoelectric element of the first pair of wedge-shaped piezoelectric elements; are caused to expand and contract 180 degrees out of phase with each other, and wherein
the first wedge-shaped piezoelectric element and the second wedge-shaped piezoelectric element of the second pair of wedge-shaped piezoelectric elements are caused to expand and contract out of phase with each other.

19. The method of claim 18, wherein the first pair of wedge-shaped piezoelectric elements and the second pair of wedge-shaped piezoelectric elements are arranged on the horn to produce a translational vibration at a distal end of the horn.

20. The method of claim 18, wherein the first pair of wedge-shaped piezoelectric elements and the second pair of wedge-shaped piezoelectric elements are arranged on the horn to produce an elliptical vibration at a distal end of the horn.
